(19) United States
(12) Patent Application Publication
(10) Pub. No.: US 2005/0100126 A1
(43) Pub. Date: May 12, 2005

(54) Computed tomography with z-axis scanning

(76) Inventors: Charles A. Mistretta, Madison, WI (US); Sarah Patch, Milwaukee, WI (US); Rostyslav Boutchko, Madison, WI (US); Jiang Hsieh, Brookfield, WI (US)

Correspondence Address: QUARLES & BRADY LLP, 411 E. WISCONSIN AVENUE, SUITE 2040, MILWAUKEE, WI, 53202-4497, US

(21) Appl. No.: 10/827,489

(22) Filed: Apr. 19, 2004

Related U.S. Application Data
(60) Provisional application No. 60/518,530, filed on Nov. 7, 2003.

(51) Int. Cl. G21K 1/12; G01N 23/00; A61B 6/00

(52) U.S. Cl. 378/15

(57) ABSTRACT

A CT imaging system includes a two-dimensional detector array and an x-ray source that both revolve around the subject during a scan. The x-ray source produces a cone beam of x-rays and the focal point of this cone beam is electrically moved to different locations along the z-axis to increase the z-axis extent of the ROI that can be imaged without patient table movement. Different focal point scan patterns are described for a variety of different clinical applications.

### CROSS-REFERENCE TO RELATED APPLICATIONS

This application claims the benefit of U.S. Provisional patent application Ser. No. 60/518,530 filed on Nov. 7, 2003 and entitled “Computed Tomography With Z-Axis Scanning”.

### STATEMENT REGARDING FEDERALLY SPONSORED RESEARCH OR DEVELOPMENT

This invention was made with United States government support awarded by the following agencies: NIH HL62425. The United States has certain rights in this invention.

### BACKGROUND OF THE INVENTION

The present invention relates to x-ray computed tomography (CT) and specifically to a CT system and method providing high-speed data acquisition.

Conventional x-ray computed tomography may employ an x-ray source collimated to produce a narrow fan-shaped beam directed along the transverse plane through a patient to be received by linear multi-element detector array.

The x-ray source and detector array may be mounted on a gantry to be rotated about a patient to obtain “projections” measuring x-ray attenuation at the different gantry angles along a slice plane though the patient. A “projection set” of projections obtained over a range of gantry rotation of 180 degrees plus the angle subtended by the fan beam, may be “reconstructed” into a tomographic image. The tomographic image shows a cross section of the patient along the slice plane.

Reconstructions using too few projections or a limited number of gantry angles may produce images with “artifacts”, typically streaks, that mar the tomographic image.

In a normal CT acquisition, multiple projection sets are obtained along different, sequential slice planes by movement of the patient within the gantry along the z-axis. The slices may be assembled to provide data for an arbitrary volume of the patient which may then be reconstructed into cross-sectional images along arbitrary planes.

The time required to collect projection data over a volume using conventional fan-beam tomography can be substantial and may preclude the use of tomography in situations where large volumes are to be monitored in real-time (for example, in dynamic contrast enhanced studies) or where there is unavoidable patient or organ motion during the acquisition time (for example, cardiac studies).

One method of increasing CT acquisition speed is by using a spiral or helical scan in which the gantry containing the x-ray source and detector is rotated continuously as the patient is moved along the z-axis. This process eliminates the need for stopping and starting the gantry and table.

Wider fan beams known as cone beams may also be used with rectangular detector arrays having, for example, 500 or 1,000 detector elements in the scan plane and sixteen detector elements disposed along the z-axis, perpendicular to the scan plane. This arrangement allows multiple slices to be collected at one time but the z-axis extent of the acquisition is very limited unless the patient table is moved during the scan.

Another approach is the electron beam CT scanner in which a rotating x-ray source is generated without mechanical motion of a gantry. Instead, as shown in U.S. Pat. Nos. 4,352,021; 4,672,649; 6,130,929; 5,491,734; 5,504,791; 5,504,791; 6,009,141; 5,172,401; and 4,158,142 the electron beam is directed to stationary anodes around the subject to produce x-rays from the desired projection angles. Although these “EBCT” systems acquire a 2D slice image very rapidly, the patient table must be moved to acquire additional slice images along the z-axis.

The commercially available scanners are thus not very fast in acquiring x-ray attenuation data over a region of interest (ROI) that is extended in the direction of table motion. One such clinical application, for example, is a contrast enhanced dynamic study of vasculature in the trunk or legs of a subject. In this clinical application it is desirable to acquire data from the extended ROI at a high frame rate so that the inflow of contrast can be observed in a series of images. This is not possible if the table must be moved during the scan to cover the entire ROI. Another clinical application is cardiac imaging in which attenuation data must be obtained over a 3D volume in very short time intervals in order to “capture” the cardiac structures at very specific cardiac phases. This cannot be done well if the scan must be repeated at two or more patient table locations to cover the ROI along the z-axis.

### SUMMARY OF THE INVENTION

The present invention is a CT system having an x-ray source that not only produces a cone beam that projects through the subject and is detected by a two-dimensional array of detector elements, but also is electronically moved along the z-axis to substantially increase the ROI without the need for table motion. As the x-ray source and 2D detector array are rotated around the gantry during the scan, the x-ray source is moved along the z-axis to acquire data from an ROI that is much larger than that acquired by a rotated cone beam.

A general object of the invention is to acquire x-ray attenuation data over a cylindrical volume in a controlled and prescribed manner. Depending on the clinical application, the physical rotation of the x-ray source around the subject and the linear electronic motion of the x-ray source along the z-axis can be controlled to implement many different acquisition strategies.

Another object of the invention is to rapidly acquire x-ray attenuation data over a very large ROI. A dynamic study can be performed from which a plurality of images are reconstructed depicting changes in the subject at a high frame rate. In a contrast enhanced CT study, for example, the in-flow of contrast can be seen in vasculature that extends a substantial distance along the z-axis.

Another object of the invention is to acquire x-ray attenuation data of the entire heart during a gated cardiac scan. The electronic scan along the z-axis enables data to be acquired from the entire heart over a very short time interval at any cardiac phase. Blurring due to cardiac motion is reduced.

The foregoing and other objects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is a pictorial view of a CT imaging system in which the present invention may be employed;

FIG. 2 is a block schematic diagram of the CT imaging system;

FIG. 3 is a partial perspective view of the CT imaging system of FIG. 1 showing the x-ray source and detector array arrangement;

FIG. 4 is a partial view of the anode assembly which forms part of the x-ray source in FIG. 3;

FIGS. 5A and 5D are pictorial representations of different scan patterns that may be prescribed using the CT imaging system of FIG. 1;

FIG. 6 is a pictorial representation of the x-ray cone beam produced at a particular moment during a scan performed by the CT imaging system of FIG. 1;

FIG. 7 is a graphic illustration of a preferred method for combining k-space data from successively acquired data sets;

FIG. 8 is a graphic illustration of a cardiac gated acquisition of data;

FIG. 9 is a partial elevation view of the collimator and x-ray source looking along the z-axis; and.

FIG. 10 is a partial perspective view of an alternative x-ray source for use in the CT system of FIG. 1.

### DETAILED DESCRIPTION OF THE PREFERRED EMBODIMENT

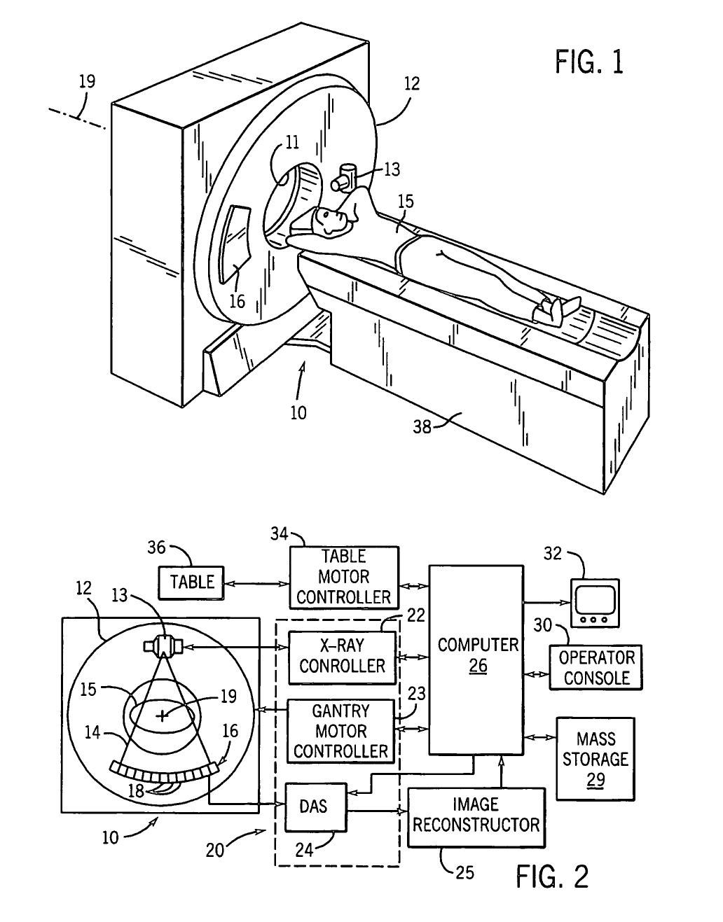

With initial reference to FIGS. 1 and 2, a computed tomography (CT) imaging system 10 includes a gantry 12 representative of a “third generation” CT scanner. Gantry 12 has an x-ray source 13 that projects a cone beam of x-rays 14 toward a detector array 16 on the opposite side of the gantry. The detector array 16 is formed by a number of detector elements 18 which together sense the projected x-rays that pass through a medical patient 15. Each detector element 18 produces an electrical signal that represents the intensity of an impinging x-ray beam and hence the attenuation of the beam as it passes through the patient. During a scan to acquire x-ray projection data, the gantry 12 and the components mounted thereon rotate about a center of rotation, or z-axis 19.

The rotation of the gantry and the operation of the x-ray source 13 are governed by a control mechanism 20 of the CT system. The control mechanism 20 includes an x-ray controller 22 that provides power and timing signals to the x-ray source 13 and a gantry motor controller 23 that controls the rotational speed and position of the gantry 12. A data acquisition system (DAS) 24 in the control mechanism 20 samples analog data from detector elements 18 and converts the data to digital signals for subsequent processing. An image reconstructor 25, receives sampled and digitized x-ray data from the DAS 24 and performs high speed image reconstruction. The reconstructed image is applied as an input to a computer 26 which stores the image in a mass storage device 29.

The computer 26 also receives commands and scanning parameters from an operator via console 30 that has a keyboard. An associated cathode ray tube display 32 allows the operator to observe the reconstructed image and other data from the computer 26. The operator supplied commands and parameters are used by the computer 26 to provide control signals and information to the DAS 24, the x-ray controller 22 and the gantry motor controller 23. In addition, computer 26 operates a table motor controller 34 which controls a motorized table 36 to position the patient 15 in the gantry 12.

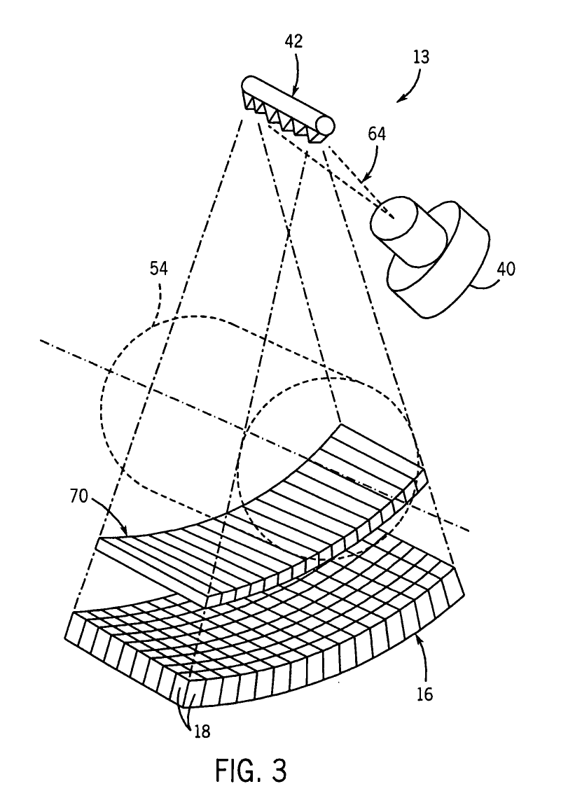

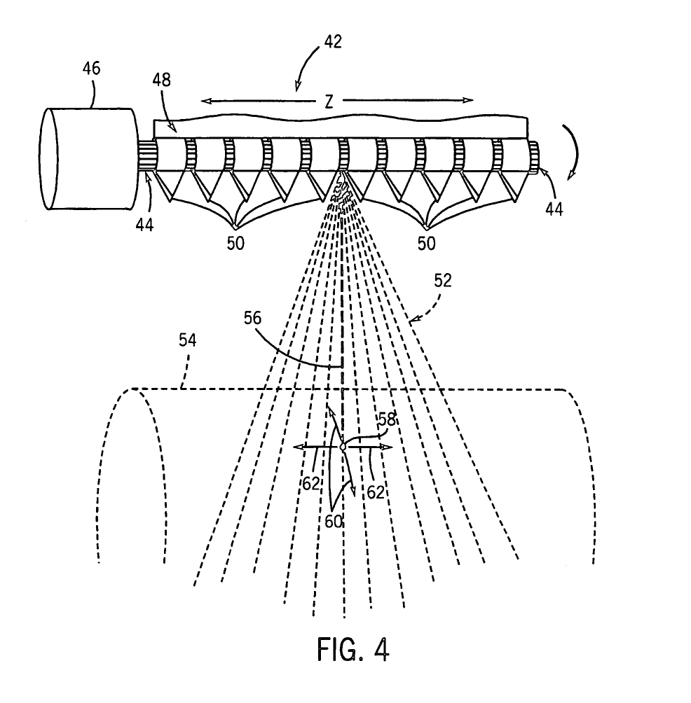

Referring particularly to FIGS. 3 and 4, the detector array 16 is a two-dimensional array of detector elements 18 arranged in rows and columns facing the x-ray source 13. The detector elements 18 may be conventional scintillation type x-ray detectors, but may also be ionization type or CZT detectors that are capable of at least a thirty frame per second readout rate. In the preferred embodiment the detector array extends along the z-axis 125 cm and it contains 2048 detector elements along the z-axis and 1024 elements in the gantry rotation direction.

Opposite the detector array 16 is the x-ray source 13, which in this preferred embodiment is comprised of an electron gun 40 and an anode assembly 42. As shown best in FIG. 4, the anode assembly is comprised of a cylindrical anode 44 made of a high-Z material such as tungsten which is mounted for rotation by a motor 46. For coronary applications the anode 44 has a length of 30 cm and it is aligned substantially parallel to the z-axis 19 that extends through the center of the gantry 12.

Mounted adjacent the rotating anode 44 is a stationary, pre-patient collimator 48. The collimator 48 is constructed of a metal which shields x-rays and includes downward extending, wedge-shaped lobes 50 that are spaced equidistantly along the length of the anode 44. Anode segments are exposed between the lobes 50 and when an electron beam strikes one of the anode segments, a cone beam of x-rays 52 is produced and extends between the adjacent collimator lobes 50 and into a cylindrical region of interest 54. As will be discussed in more detail below, the electron beam can be moved quickly to strike any of the exposed anode segments, and the cone beam 52 can thus be moved electronically along the z-axis to different locations. The anode assembly 42 is also rotated around the region of interest 54 as the gantry 12 rotates. The cone beam 52 which it produces can thus intersect the region of interest 54 at any view angle. As shown in FIG. 4, if the central ray of the cone beam 52 is represented by dashed line 56 and the intersection of this central ray with the cylindrical ROI surface is a scan point 58, a scan of the ROI 54 can be defined by the path of the scan point 58. In other words, a scan pattern of the ROI 54 is defined by movement of the scan point 58 circumferentially around the cylindrical ROI 54 as indicated by arrows 60 and its movement along the axial, z-axis as indicated by arrows 62.

Referring again to FIG. 3, the anode assembly 42 and the electron gun 40 are enclosed in an airtight housing (not shown) that is evacuated. The electron gun 40 is positioned such that an electron beam 64 from the gun 40 strikes the anode assembly 42 to produce an x-ray cone beam directed toward the ROI 54. The electron gun 40 includes an electron source (not shown), for example, a heated filament, to produce electrons that are formed into an electron beam 64 and drawn toward the anode assembly 42 by an accelerating voltage maintained between the anode assembly 42 and the electron gun 40. Electrostatic plates or magnetic yokes (not shown) within the electron gun 40 deflect the electron beam 64 and enable it to be electronically steered to any location along the length of the anode assembly 42. The electron beam 64 can thus be quickly steered to impact any one of the anode segments disposed along its z-axis extent. This deflection provides almost instantaneous z-axis movement of the x-ray cone beam focal point and the only limitation on the rate at which the cone beam focal point is moved to different anode segments along the z-axis is the need to dwell long enough at any one segment to enable sufficient x-rays to be produced for the detector elements 18 that are used. Detector elements used in currently available CT scanners can acquire separate attenuation measurements at a rate of thirty per second, but it is anticipated that his measurement rate will increase substantially in the coming years. A 912×266 element detector array that reads 900 frames per second is described, for example, by Saito et al, “Large Area 2D Detector For 3D CT 4DCT” Medical Imaging 2001: Physics of Medical Imaging, Proceedings of the SPIE Vol. 4320 (2001).

While a single electron gun 40 is used in the preferred embodiment and controlled by the x-ray controller 22 (FIG. 2), it should be apparent to those skilled in the art that separate electron guns may be used for each z-axis anode segment in the anode assembly 42. Rather than steering a single electron beam to scan along the z-axis, in this alternative embodiment the separate electron beams are switched on an off as prescribed to move the x-ray cone beam focal point along the z-axis.

Referring still to FIG. 3, to shield the detector array 16 from external radiation and to thereby reduce the total x-ray dose needed to provide the desired SNR, a collimator 70 is disposed between the ROI 54 and the detector array 16. The collimator grid 70 is comprised of a set of spaced metal walls that extend along the z-axis and are aligned with the boundaries between adjacent detector elements 18. These walls absorb radiation that enters from external sources and blocks this radiation from striking the detector elements 18. The objective is to admit x-rays from the x-ray source 13 while blocking random radiation. In addition to reducing the required x-ray dose for an image of prescribed SNR, the reduced x-ray exposure means that the cone beam can be scanned along the z-axis at a higher rate.

As shown in FIG. 9, the collimator 70 is fabricated from a slab of radiation absorbent material, such as tungsten, to define a plurality of channels 71 that extend along the z-axis and allow x-rays to pass through to the detector 16. The channels 71 are “focally aligned”, meaning that each channel 71 is directed to receive x-rays from the same x-ray source 42.

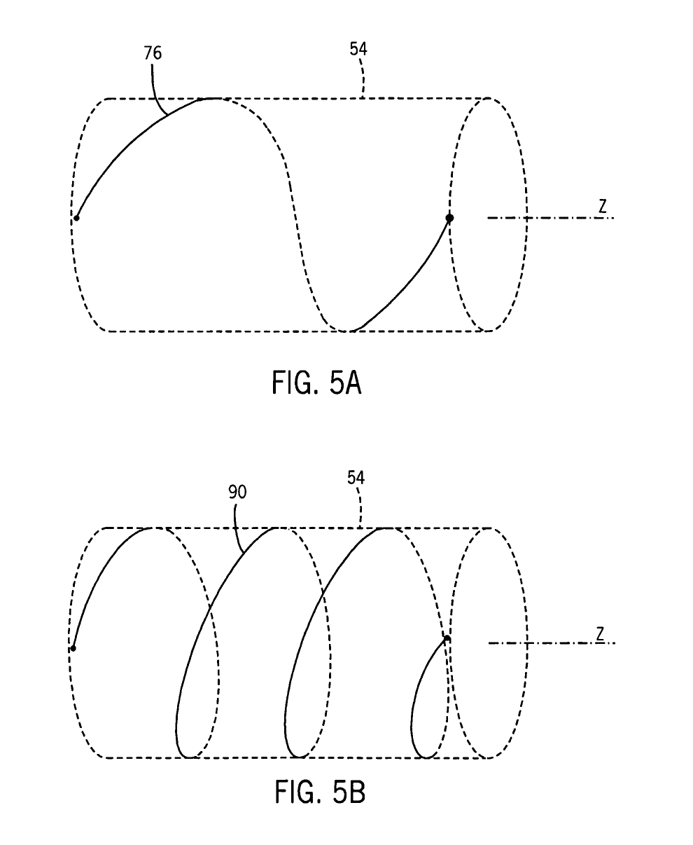

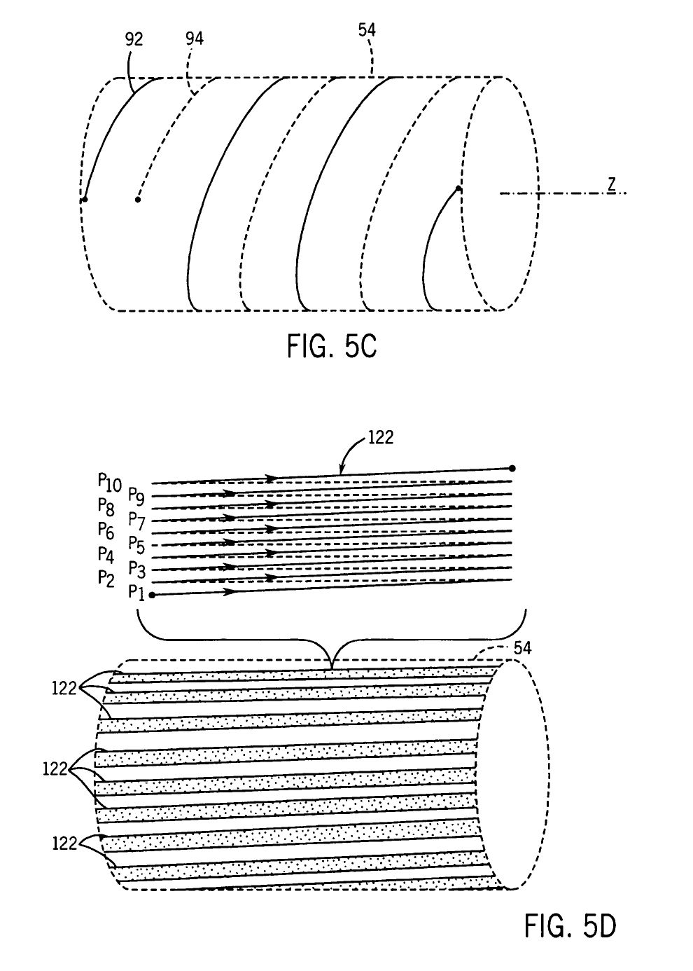

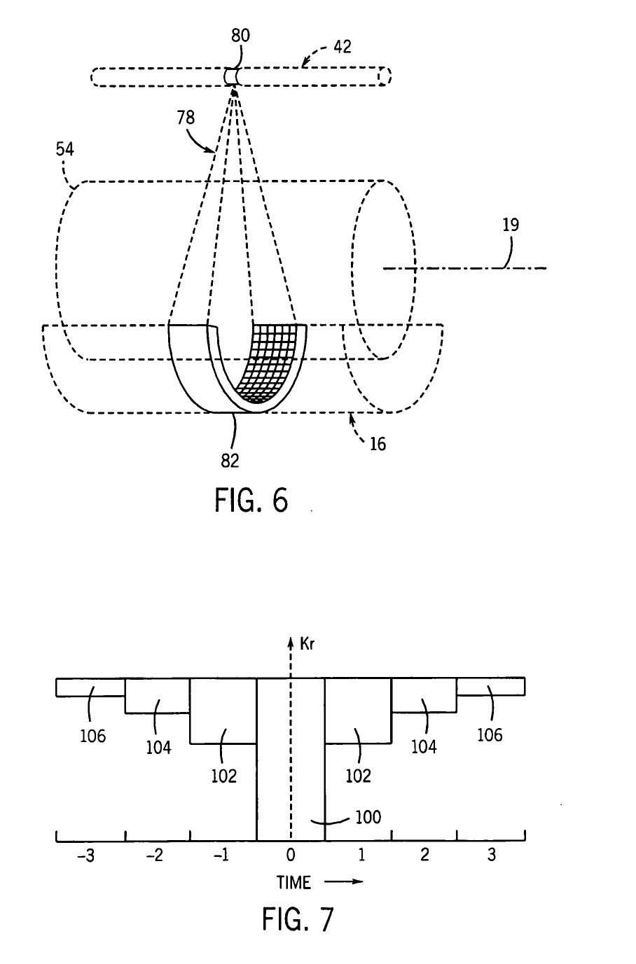

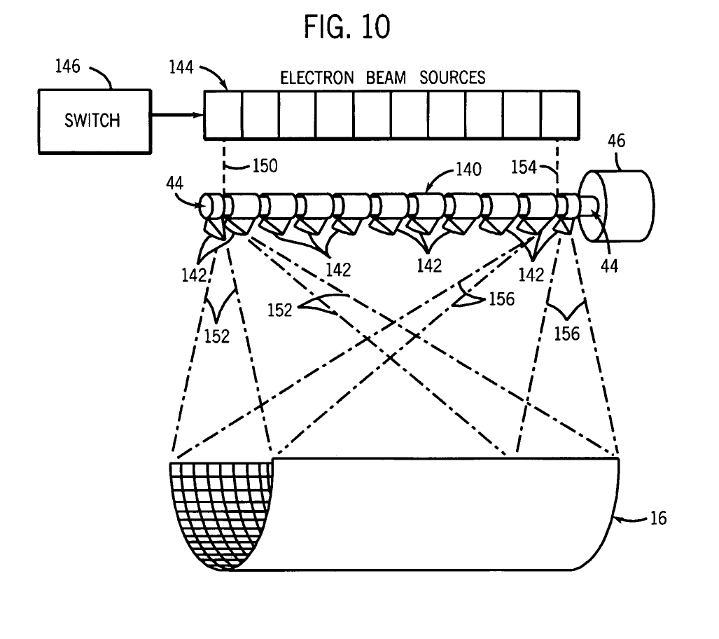

Referring particularly to FIGS. 2 and 5A, the x-ray controller 22 and the gantry motor controller 23 may be operated according to a prescribed scan to move the scan point 58 in a variety of different patterns. The particular scan pattern chosen will depend on the clinical application. If the x-ray cone beam is moved along the z-axis such that it travels the entire z-axis extent in one revolution of the gantry 12, a simple spiral pattern 76 is produced by the scan point 58. At any given moment during this scan a cone beam of x-rays 78 is produced from a focal point at one of the segments 80 on the anode assembly 42 and this cone beam 78 is detected by the detector array 16. In one preferred embodiment the detector array 16 has 2048 elements in each column along the z-axis and the cone beam intersects a segment 82 of ______ elements as shown in FIG. 6. In another preferred embodiment the cone beam 78 intersects all 2048 elements in each column of the detector array 16 as shown in FIG. 10. Thus, more than one slice of data is being acquired at all times.

It should be apparent, however, that if the scan pattern of FIG. 5A is used in which the entire z-axis extent of the anode array 42 is utilized during a single gantry revolution, an insufficient number of view angles will be acquired for any given slice through the ROI 54. This is due to the fact that for any given slice along the ROI 54 the cone beam 78 does not impinge on the detector elements 18 in that slice plane at a sufficient number of view angles. In other words, the z-axis movement of the cone beam 78 moves past the detector elements 18 in any given slice before they see enough different view angles. As a result, the acquired attenuation data for each slice is highly undersampled.

One solution to the undersampling problem is to use a scan pattern in which multiple gantry rotations are performed during the movement of the scan point 58 along the z-axis. Such a scan pattern 90 is illustrated in FIG. 5B where three revolutions of the gantry 12 occur as the scan point 58 traverses the z-axis. This scan pattern will triple the number of view angles acquired at any given slice, but the scan time is also tripled.

Referring particularly to FIG. 10, an alternative embodiment of the x-ray source 13 includes a rotating anode 44 which is rotated by motor 46. A stationary, pre-patient collimator 140 surrounds the anode 44 and includes eleven downward extending, wedge-shaped lobes 142 that are spaced equidistantly along its length. Anode segments are exposed between the lobes 142 and when an electron beam strikes one of these anode segments, a cone beam of x-rays is produced that strikes the detector array 16.

Unlike the first embodiment described above, this second embodiment employs separate electron beam sources 144 for the ten separate anode segments. The electron beam sources 144 are each switched on and off during a scan by an electron switch 146 that is operated by the x-ray controller 22 (FIG. 2). The electron beam produced by each source 144 need not be deflected, or moved during the scan, but rather, it is turned on by the switch 146 when the scan pattern calls for the cone beam focal point to emanate from its associated anode segment.

Another distinction between the first and second embodiment is the shape of the cone beam that is produced. As shown best in FIG. 6, in the first embodiment the cone beam 78 fans out along the z-axis direction to intersect a segment 82 of less than all the detector elements 16. The specific shape of this cone beam 78 is determined by the shapes of the lobes 50 (FIG. 4) on the pre-patient collimator 48. In the second embodiment shown in FIG. 10, the lobes 142 of the pre-patient collimator 140 are shaped to enable the cone beam to intersect the entire detector array. Thus, when the focal point emanates from the left-most anode segment by energizing it with an electron beam indicated by dashed line 150, the resulting cone beam indicated by lines 152 is skewed far to the right to intersect detector elements throughout the array 16. The same is true when the right-most anode segment is energized by an electron beam indicated by dashed lines 154. In this case the resulting cone beam indicated by lines 156 is skewed far to the left to intersect all the detector elements in array 16. The advantage of this second embodiment is that all the detector elements in array 16 are employed to acquire data from the subject throughout the scan. Unlike data acquired with a fan beam which intersects only a single row of detector elements at a right angle to the z-axis, with this second embodiment more attention data is acquired and most of it is at projection angles that are not perpendicular to the z-axis.

Dynamic Study

Another scanning strategy that may be used is to interleave the scan patterns during sequential acquisition of image frames during a dynamic study. Referring to FIG. 5C, such an interleaved acquisition may include a first image acquisition from the ROI 54 using a first scan pattern 92 which traverses the z-axis in three gantry revolutions. This is followed by a second image acquisition from the ROI in which a similar scan pattern 94 is employed, but it is shifted along the z-axis from the first scan pattern 92 to move the scan point 58 to interleaved locations.

The interleaved acquisition is particularly useful to perform a dynamic study where images are acquired quickly in succession in order to see changes that occur in the subject. A contrast enhanced dynamic study is an example clinical application. In this application a plurality of interleaved scan patterns are selected and each acquires an image at the desired frame rate. In this example, each scan pattern is completed in three gantry revolutions to provide a frame time of 1.5 seconds and because successive images are acquired at different, interleaved view angles, successively acquired data sets contain data measured at different view angles.

Because successive images are acquired with interleaved scan patterns, image artifacts can be reduced or suppressed by filling in missing data with data acquired from a successive image acquisition. For example, if image artifacts restrict the clinical usefulness of one image in a series of images in dynamic study, projections from temporally adjacent image data sets may be combined with its acquired data set to add more projection angles. Artifacts due to undersampling will be reduced in the reconstructed image. While such a straight forward combination of acquired data from successive, interleaved data sets may suppress image artifacts, it may also reduce the time resolution of the image. In other words, two combined data sets may have a frame time of one second rather than a frame time of 0.5 seconds. This may or may not be a problem depending on how rapidly the changes are occurring in the subject at this point in the dynamic study.

A more sophisticated method may also be used to combine interleaved data sets without significantly reducing time resolution. With this method each interleaved, undersampled acquired data set is first converted to a k-space data set. This can be done for example by reconstructing the image in a conventional manner and then Fourier transforming the image to k-space. It is a characteristic of k-space data sets acquired with three-dimensional projections that the center of k-space becomes fully sampled before peripheral k-space. It is also a characteristic of k-space that the peripheral k-space data does not significantly impact the appearance of large shapes in the final image. Therefore, peripheral k-space data can be shared between successive, interleaved k-space data sets to fill up, or provide more fully sampled k-space data sets without changing the effective time resolution of the succession of images. Each combined k-space data set is then used to reconstruct a 3D image by performing a 3D Fourier transformation. The resulting images have reduced image artifacts and the time resolution is not substantially changed because the changes in large shapes that occur during the dynamic study are not affected by sharing peripheral k-space data.

In the preferred embodiment only two different interleaved scan patterns are employed during the dynamic study. This enables the data from any two successive data sets to be combined as described above. In some clinical applications it may be preferable to use more than two different interleaved scan patterns and data may be combined from more than two successively acquired data sets. To avoid unduly reducing the time resolution of the dynamic study the peripheral k-space data sharing method should be used when combining data from more temporally remote data sets. For example, peripheral data extending closer to the center of k-space may be combined from temporally adjacent data sets, whereas peripheral k-space more remote from the center of k-space may be combined from data sets acquired more remote in time from the image frame being reconstructed. This method of combining k-space data is illustrated in FIG. 7, where the vertical axis, kᵣ, is the radial distance of k-space sample data from the center of k-space and the horizontal axis is data acquisition time relative to the acquisition time of the current image frame being reconstructed. In this example, seven different interleaved scan patterns are used such that unique k-space samples are produced for any seven successively acquired data sets. When reconstructing an image using any one data set, all of the k-space data is used from that data set as indicated at 100. This is combined with substantial peripheral k-space data 102 from the two temporally adjacent data sets and successively less peripheral k-space data 104 and 106 from the more temporally remote acquired data sets.

Cardiac Study

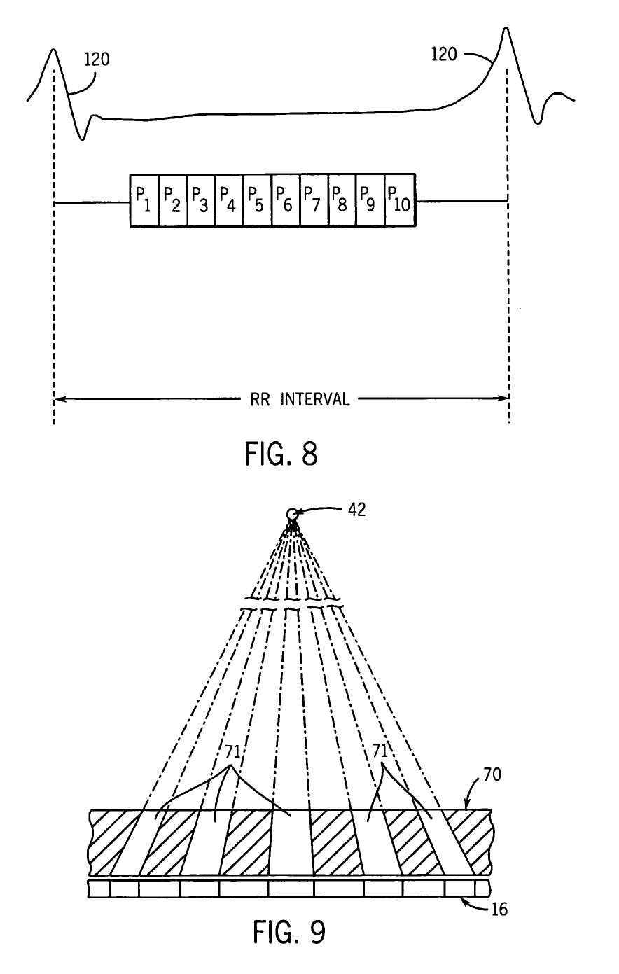

Another clinical application of the present invention is in cardiac gated studies of the heart. Because the heart is moving rapidly a gating signal is used to trigger the acquisition of views at specific phases of the cardiac cycle. Referring particularly to FIG. 8, three-dimensional images of ROI 54 are acquired which depict the heart at a succession of cardiac phases P₁-P₁₀ following the receipt of a cardiac trigger signal 120. The attenuation data for each cardiac phase image P₁-P₁₀ is acquired in segments over a very short 10 msec time interval after each cardiac trigger signal 120. Over a 20 second patient breath hold at a heart rate of 60 beats/second, therefore, this enables a total of twenty segments to be acquired.

Referring particularly to FIG. 5D, the scan pattern for this acquisition is comprised of twenty bands 122 of acquisition activity spaced substantially evenly around the ROI 54. This is achieved by revolving the gantry at a rate in which it completes one revolution in a 20 second breath hold. As the gantry slowly revolves, a band 122 of acquisition activity will occur after each cardiac trigger signal 120, during which one segment of data will be acquired for each phase image P₁-P₁₀. In this embodiment only ten cardiac phase images are acquired and there is considerable idle time between the bands 122 during which the system waits for the start of the next cardiac cycle. If more cardiac phase images are acquired, these acquisition bands become wider and the spacing between them is reduced.

Referring still to FIG. 5D, each segment of attenuation data is acquired by scanning along the z-axis as fast as possible. That is, the x-ray focal point is moved to successive points on the anode 44 as fast as possible. As described above, the rate at which this can be achieved is determined by the amount of photons needed by the detector elements 18 to achieve the desired image SNR. With current technology this enables ten different exposure points to be acquired along the z-axis in a 10 msec cardiac phase interval. Each acquisition activity band 122 is comprised of moving the scan point 58 as fast as possible in a raster scan pattern to acquire a z-axis line of ten focal point exposures for each of the ten cardiac phase images. While only ten cardiac phases are acquired in this embodiment, many more cardiac phases can be acquired if desired. As many as 50 to 100 cardiac phases can be acquired, but it is likely that fewer will be acquired in order to reduce the exposure to x-rays.

At the completion of the scan, data for each cardiac phase image has been acquired. It includes data that covers the entire z-axis extent of the ROI 54 and with view angles distributed substantially evenly around the ROI. This 3D attenuation data set is used to reconstruct a 3D image using conventional cone beam reconstruction methods.

It should be apparent that many other scan patterns may be implemented using the present invention to accommodate the particular needs of other clinical applications. Also, in most applications no table motion is needed due to the z-axis extent of the electronically moved x-ray cone beam, but there still may be clinical applications where some table movement is employed. It should also be apparent that other types of x-ray sources may be employed, such as transmission type x-ray sources rather than the reflective type x-ray sources employed in the preferred embodiment.

1. A method for acquiring image data with a computer tomography imaging system, the steps comprising:
a) producing a cone beam of x-rays with an x-ray source and directing it into a region of interest in accordance with a prescribed scan pattern; and
b) detecting x-rays in the cone beam after they have passed through the region of interest;
wherein the x-ray source is mechanically moved around the region of interest when performing the prescribed scan pattern and a focal point of the cone beam of x-rays is electronically moved to positions along an axial dimension of the region of interest when performing the prescribed scan pattern.

2. The method as recited in claim 1 in which steps a) and b) are repeated to acquire an additional set of image data with a second scan pattern that is interleaved with the prescribed scan pattern.

3. The method as recited in claim 1 in which the x-ray source is comprised of an electron gun and an anode and the cone beam of x-rays is produced by directing the electron beam at a focal point on the anode, and wherein the electronic movement of the focal point of the cone beam of x-rays is performed by electronically moving the electron beam.

4. The method as recited in claim 1 in which the prescribed scan pattern includes moving the x-ray source around the region of interest and periodically electronically moving the x-ray source along the axial dimension in response to a cardiac trigger signal.

5. The method as recited in claim 4 in which the x-ray source is moved once around the region of interest during the performance of the prescribed scan pattern and the x-ray source is electronically moved a plurality of times along the full extent of the axial dimension a plurality of times in response to each cardiac trigger signal.

6. The method as recited in claim 1 in which the x-ray source is comprised of a set of separate x-ray sources disposed along the axial dimension of the region of interest and the electronic movement of the focal point of the cone beam of x-rays is performed by switching the separate x-ray sources on and off during the prescribed scan pattern.

7. A computer tomography imaging system which comprises:
a table for supporting a subject in a cylindrical region of interest disposed along an axis;
an x-ray source for producing a cone beam of x-rays directed into the cylindrical region of interest;
a two-dimensional array of detectors disposed around a portion of the cylindrical region of interest and oriented to detect x-rays in the cone beam after they pass through the region of interest;
a gantry for supporting the x-ray source and two-dimensional array of detectors and for rotating them around the cylindrical region of interest in a plane perpendicular to the axis;
means for electronically moving a focal point of the cone beam of x-rays to positions along the direction of the axis;
means for directing the rotation of the gantry and the electronic axial movement of the cone beam focal point in accordance with a prescribed scan pattern; and
means for acquiring signals produced by detected x-rays during performance of the prescribed scan pattern and reconstructing an image therefrom.

8. The computer tomography imaging system as recited in claim 7 in which the two-dimensional array of detectors extends substantially the entire axial length of the cylindrical region of interest.

9. The computer tomography imaging system as recited in claim 7 in which the x-ray source includes an electron gun that produces an electron beam that strikes an anode to produce the cone beam of x-rays and the means for electronically moving the cone beam focal point along the axial direction moves the electron beam.

10. The computer tomography imaging system as recited in claim 7 which further includes a collimator disposed between the two-dimensional array of detectors and the cylindrical region of interest which is operable to reduce radiation from sources other than the x-ray source reaching the two-dimensional array of detectors.

11. The computer tomography imaging system as recited in claim 10 which includes a second collimator disposed between the x-ray source and the cylindrical region of interest which is operable to limit the axial extent of the cone beam of x-rays reaching the region of interest.

12. The computer tomography imaging system as recited in claim 7 in which the x-ray source includes a set of separate x-ray sources disposed along the direction of said axis and the means for electronically moving the focal point includes a switch connected to turn the separate x-ray source on and off.

13. The computer tomography imaging system as recited in claim 7 which includes a collimator disposed between the x-ray source and the cylindrical region of interest to shape the cone beam of x-rays.

14. The computer tomography imaging system as recited in claim 13 in which the cone beam of x-rays is shaped to intersect substantially all the detectors in said two-dimensional array of detectors when produced from any of said focal point positions along the direction of the axis.

15. The computer tomography imaging system as recited in claim 13 in which the cone beam of x-rays is shaped to intersect a segment of the detectors in said two-dimensional array of detectors and said segment includes substantially less than all the detectors in said array of detectors.

16. A method for producing an image with a computer tomography imaging system, the steps comprising:
a) producing a cone beam of x-rays with an x-ray source and directing it into a region of interest;
b) detecting x-rays in the cone beam after they have passed through the region of interest;
c) controlling the scan pattern of the x-ray source by moving it around the region of interest and moving it electronically to positions along an axial dimension of the region of interest to acquire a first attenuation data set with a first spiral scan pattern;
d) repeating step c) to acquire additional attenuation data sets with spiral scan patterns that interleave;
e) transforming the acquired attenuation data sets to a corresponding series of k-space data sets;
f) combining k-space data from temporally adjacent k-space data sets; and
g) reconstructing an image from the combined k-space data.

17. The method as recited in claim 16 in which the combined k-space data is formed by combining all the k-space data from one of said k-space data sets with peripheral k-space data from another, temporally adjacent k-space data set.

18. The method as recited in claim 17 in which a series of images are produced by combining all of the k-space data from respective ones of the k-space data sets with peripheral k-space data from temporally adjacent k-space data.

19. A method for producing an image with a computer tomography imaging system, the steps comprising:
a) producing a cone beam of x-rays with an x-ray source and directing it into a region of interest;
b) detecting x-rays in the cone beam after they have passed through the region of interest;
c) controlling the scan pattern of the x-ray source by moving it around the region of interest and moving it electronically to positions along an axial dimension of the region of interest to acquire a first attenuation data set with a first spiral scan pattern;
d) repeating step c) to acquire additional attenuation data sets with spiral scan patterns that interleave; and
e) reconstructing an image by combining all of the data from one of said attenuation data sets with a part of the data from a temporally adjacent attenuation data set.

20. The method as recited in claim 19 in which a series of images are produced by combining all of the data from respective ones of said attenuation data sets with less than all of the data from temporally adjacent attenuation data sets.

21. The method as recited in claim 19 in which said attenuation data sets are processed before being combined.
